(19) United States
(12) Patent Application Publication
(10) Pub. No.: US 2003/0164172 A1
(43) Pub. Date: Sep. 4, 2003

(54) Method and apparatus for guiding a surgical instrument

(76) Inventors: Nicole Jane Chumas, Lane Harrogate (GB); Paul Dominic Chumas, Lane Harrogate (GB)

Correspondence Address: Charles D Gunter Jr, Bracewell & Patterson, Suite 1600, 201 Main Street, Ft Worth, TX, 76102, US

(21) Appl. No.: 10/297,523

(22) PCT Filed: Jun. 8, 2001
(86) PCT No.: PCT/GB01/02523

(30) Foreign Application Priority Data
Jun. 9, 2000 (GB) 0014059.0

(51) Int. Cl. A61B 17/00; A61B 19/00

(52) U.S. Cl. 128/898; 606/1

(57) ABSTRACT

The present invention relates to a method and apparatus which provides proactive guidance to a surgeon for guiding the tip of a surgical instrument along a previously defined optimum path within the body of a mammal to a tumour or other target within the body. This path is subdivided into a plurality of sections extending from one intermediate point to another and an image is formed on the exposed surface of the body of the mammal upon which the surgeon is operating. The image is focused below the exposed surface and at the next intermediate point to be traversed along the path. The image provides the surgeon with vector information as to where he should direct the tip of the instrument. The image is preferably three or more overlapping dots of light from lasers which are directed at the intermediate point by motorised mountings under the control of a computer, the splay of the dots of light giving the surgeon the vector information. By providing the image focused at the intermediate point, the surgeon is provided with a simple visual vector to follow whose direction is continuously updated as the surgeon moves the tip of the scalpel or other surgical instrument towards the intermediate point.

The present invention relates to a method and an apparatus for use in that method, notably to a method for proactively guiding the operative tip of a surgical instrument during surgery.

### BACKGROUND TO THE INVENTION

Where surgery is to be performed upon a person, it is customary to take a plurality of X ray or other images showing the internal structure of the body to identify the shape and location of the specific problem upon which the surgery is to be carried out. For example, where a tumour within the brain is to be removed, a series of X ray, ultrasound or MRI images is taken of the brain to provide images which show the form and location of the tumour within the skull of the patient. If computer-assisted surgery is to be undertaken, fiducial markers are usually secured to the skin or screwed into the skull of the patient prior to image acquisition to provide datum points against which the images can be related. Typically, a number of images are taken from a plurality of directions and/or at different axial locations relative to the body or head of the patient to generate a plurality of slice images of the head. Those images are subjected to image processing using a computer and appropriate programs so as to build up a three-dimensional computer image. This image can be displayed upon a visual display unit and the displayed image can be rotated, enlarged or otherwise manipulated to assist the surgeon to identify the exact shape and location of the tumour within the skull to provide datum points against which the internal structures of the brain can be related.

This technique can be applied to other features within the body, for example detection of thromboses in blood vessels, and to other procedures to be carried out on the body. For example, it can be applied in an exploratory investigation of a potential tumour, or for directing the insertion of implants, for example pins or plates in spinal or orthopaedic surgery. The technique can also be applied to mammals other than humans, for example horses or other domestic animals. However, for simplicity, the invention will be described hereinafter in terms of tumours within the brain of a human patient.

Having determined the shape and location of the tumour, the surgeon can then determine the optimal path through the skull and brain to reach the tumour with minimum disruption of or damage to adjacent tissues. During the surgical procedure itself, the patient's head is clamped or otherwise secured firmly in position. The positions of the fiducial markers in the patient's head are established with relation to the computerised image by touching the markers with a wand or probe which carries an LED or other indicator, whose position can be detected by a series of CCD or other cameras or sensors mounted in the ceiling or other suitable fixed points in the operating theatre. By triangulation of the positions of the fiducial markers, a computer can determine the position and location of the patient's head and relate this to the computerised image of the internal structure of the brain and the tumour. Since the fiducial markers are often difficult to detect during the surgical procedure, a reference arc or similar device is usually attached to the clamp securing the patient's head in position. This arc carries LEDs or other emitters whose position can be detected by the cameras and related by the computer to the computerised image and the position of other equipment used by the surgeon. Thus, the position and orientation of the patient's head can be determined at any time during the surgical procedure and the computerised image of the brain and tumour and its display corrected if the head is moved. This registration procedure enables the position of a trackable instrument, for example a probe, within the skull and brain to be related to the computerised image. The surgeon determines that he is following the optimal path by detecting the position to which any incision made by him has penetrated within the skull or the brain. The surgeon can then relate this position to the computer image generated in the initial survey of the patient's head so as to determine where he should next direct the surgical instrument so as to arrive at the tumour.

To do this, it is necessary for the surgeon to withdraw the surgical instrument he is using to penetrate the skull or brain and to insert a probe or wand into the incision. The distal tip of the marker probe carries an LED or other device by which the tip can be detected by a series of fixed sensors, for example infra red cameras or sensors secured to the ceiling of the operating theatre or other fixed locations, which are spaced apart from one another. These sensors provide a triangulated position detection of the tip within the brain. The relevant portions of the computerised images are displayed on the computer screen together with the location of the probe within the brain. As a result the surgeon can determine how the actual path of his incision relates to the optimal path determined from the initial survey.

However, such a technique requires that the surgeon interrupts the surgical procedure, remove the scalpel or other surgical instrument from the incision and insert the marker probe or wand into the incision he has made. This is disruptive for the surgeon and carries the risk of damage to the brain or other tissue by the repeated removal and insertion of the probe and the scalpel or other surgical instrument. Furthermore, in order to determine the position of the marker probe, the surgeon must look away from the patient's head and direct his attention at a VDU or other display device to view the image which relates the position of the marker probe or wand to the structure of the brain. The surgeon must then make a topographical assessment of the direction in which to move the tip of the scalpel so as to follow the optimal path to the tumour. This is disruptive and tiring for the surgeon and is open to errors.

Most brain surgery is done using a microscope, through which the surgeon observes the area of the brain upon which he is operating and the operative tip of the surgical instrument, which he is using to perform the surgical procedure. It has been proposed that the chassis which carries the optical elements of the microscope be provided with means by which the position of the microscope can be detected and then related to the computerised image of the patient's skull. Since the operative tip of the surgical instrument will usually be located at the focal point of the microscope, the position of the instrument tip can be calculated from a knowledge of the position and orientation of the microscope chassis and the focal length of the microscope without the need to remove the instrument from the patient and the insertion of a marker probe or wand. It has been proposed that two laser beams carried by the microscope chassis be directed onto the head of the patient so that the beams converge at the focal point of the microscope. When the surgeon moves the microscope so that the beams converge at the point upon which he is operating, the computer monitoring the movement of the microscope can relate that point to the computerised image of the brain. This technique is used to determine the position of the tip of the surgical instrument within the brain without the need to remove the surgical instrument and replace it with a marker probe. However, the surgeon must still relate the position of the tip of the instrument to the desired path which he is to follow to the tumour. This requires that he look away from the microscope and refer to a VDU for the display of the computerised image and the position of the instrument tip relative thereto so that he can then estimate the direction in which he should next direct the tip of the scalpel to follow or re-gain the desired path. This is disruptive for the surgeon and requires that he exercise topographical interpretation of the information presented to him, which is tiring.

It has been proposed that the computerised image of the brain be displayed as an overlaid image in the optical path of the microscope. The surgeon can then view the computerised image and assess the relative positions of the tip of the instrument and the tumour within the brain without the need to divert his eyes from the microscope. However, since this must usually be done a plurality of times during a single surgical procedure, this is still disruptive and tiring for the surgeon. Furthermore, although the computerised image shows the location of the tumour, this method still requires the surgeon to exercise topographical interpretation of the images presented to him in order to estimate where he should next direct the surgical instrument.

It has also been proposed to provide the microscope with motors, which move it in three dimensions under the control of the computer handling the computerised image of the brain and tumour. The surgeon moves the microscope so as to follow the movement of the tip of the scalpel and maintain the tip at the focal point of the microscope. This movement can be detected by the cameras or other sensors on the operating theatre ceiling or by movement sensors on the microscope. As a result, the computer can determine at any time the location of the focal point of the microscope (and hence the tip of the scalpel located at that point) relative to the computerised image and the tumour within that image. By actuating a switch, the surgeon can display the location of the tip of the scalpel relative to the tumour or the optimal path to the tumour so that he can determine that he is following the correct path to the tumour. However, the surgeon still has to exercise topographical skills in assessing where next to direct the tip of the scalpel.

Such methods provide retroactive information as to where the tip of the surgical instrument is located relative to the desired path it is to follow. They do not provide direct guidance as to how the surgeon should move the tip of the surgical instrument so as to reach the tumour or other target within the brain. In order to provide such a proactive guidance to the surgeon, it has been proposed that the surgeon cause the microscope to move from the position at which it observes the exposed surface of the brain at the point where the instrument has reached on its path to the target to a position at which its focal point is located at the tumour as determined from the computer memory store of the co-ordinates of the target. This will indicate the general direction in which the surgeon should move the tip of the scalpel to arrive at the tumour or other target. However, such a technique requires the use of a complex and expensive motorised microscope and repeated switching between modes for locating the tip of the scalpel and the location of the tumour. This is complex and tiring for the surgeon and does not give a simple and continuous guidance as to where to direct the tip of the scalpel. Furthermore, such guidance does not accommodate any changes in direction in the optimum path between the target and the position which the tip of the instrument has reached along the desired path. It gives a straight line indication of the target relative to the position at which the tip of the scalpel is located and the surgeon then has to review the computerised image of the brain to determine whether there are obstacles, such as blood vessels, to following such a straight line path and to make a topographical estimate of the route to be followed.

There thus still exists a need to provide the surgeon with a simple proactive guidance of the correct path to follow during surgery without the need to interrupt the surgical procedure, without the need for additional mental effort from the surgeon, without the need to insert and remove instruments repeatedly during the surgical procedure, and without the need for the surgeon to look away repeatedly from the microscope to observe a VDU or other display so as to determine the position of the incision relative to the tumour or other target.

We have devised a method and apparatus which reduces the above problems. Furthermore, the method and apparatus of the invention can be applied to surgical procedures which do not require the use of a microscope as with the prior art techniques described above. Thus, the invention can be applied to general surgery, for example in the spine, or to assist insertion of metal implants or the like, where the direction and location of anchoring screws or bolts can be guided. By providing a simple vector guidance to the surgeon as to where he should next direct the tip of the scalpel, the surgeon can be provided with simple guidance continuously throughout the surgical procedure without the need to interrupt his concentration on the movement of the tip of the scalpel.

### SUMMARY OF THE INVENTION

Accordingly, the present invention provides a method for proactively directing the movement of the operative tip of a surgical instrument during a surgical procedure being carried out on a mammal along a predetermined path within the body of the mammal from an exposed surface of the body of the mammal via at least one intermediate point along that path to a desired target within the body of the mammal, characterised in that the method comprises:

a. directing a detectable image onto the exposed surface of that portion of the mammal upon which the surgical procedure is being carried out, which image is focused at a point below that exposed surface, which point is one of the intermediate points to which the operative tip of the surgical instrument is to be transported along the path to the target during the procedure; and

b. causing the tip of the surgical instrument to follow the image to its focus and thus to that intermediate point along the path to the target, which creates a second exposed surface at that intermediate point.

The second exposed surface may be at the target so that the path between the first exposed surface and the target is a straight line. However, where the path to the target passes through one or more intermediate points, the method of the invention comprises the further steps of:

c. re-focusing the image below that second exposed surface at the next intermediate point in the path to the target; and

d. causing the tip of the surgical instrument to follow the image from the second exposed surface to the next intermediate point; and

e. repeating, if necessary, steps c and d until the target is reached.

In the method of the invention, the image projected onto the exposed surface of the brain or other organ upon which a surgical procedure is being carried out provides the surgeon with vector information directing the surgeon to the point of focus of the image. The point of focus of the image corresponds to the location of the tumour or other target within the brain or to a way point along the optimum path from the point of entry into the skull or body of the patient to the tumour or other target within the body of the patient. The optimum path which the surgeon is to follow is determined from the initial computerised images of the patient's skull as with conventional surgical procedures. If the path from the initial point of entry into the skull to the tumour is a straight line, then the focus of the image can be at the tumour. However, it may be necessary to follow a tortuous path in order to avoid damage to other structures within the skull, for example blood vessels. In this case the path to be followed can be defined as a series of shorter straight line paths between intermediate points along the overall path. In this case each intermediate point will be a focal point for the image projected onto the exposed surface of the brain. The images projected are presented as a series of separate images one after the other as the tip of the surgical instrument progresses from one intermediate point to the next along the desired path. The surgeon is thus presented at each stage of the operation with a guide as to where the tip of the instrument should be directed to achieve the next intermediate point.

Since the directional, or vector information is presented to the surgeon on the surface upon which he is operating, he does not have to look away from the microscope. Since the information relates to where the tip of the surgical instrument should be directed, there is no need for the surgeon to determine where the tip of the instrument is, provided that he has followed the vector information to that moment in the surgical procedure. However, if desired, information relating the actual position of the tip of the instrument can be provided to the surgeon to verify that he has not strayed excessively from the optimum path. Such positional information can be provided by any suitable technique. For example, the surgeon can manually focus the image on the site at which the tip of the surgical instrument is currently working and confirm that this site lies upon the intended path by suitable programming of the computer handling the image data. However, the information will be described hereinafter in terms of the provision of solely vector information.

As indicated above, the invention can be applied to a wide range of surgical or investigative procedures performed on humans and other mammals, for example the dissection of a limb or the spine so as to enable an implant to be inserted. For convenience, the invention will be described hereinafter in terms of a tumour within the brain. Furthermore, the procedure need not involve the cutting of tissue, but may be, for example, the separation of lobes of the brain using retractors or paddles until the tumour is reached and exposed, at which point another instrument such as a laser or ultrasonic aspirator may be used to remove the tumour. For convenience, the term surgical instrument will be used herein to denote any instrument used to penetrate or investigate the body, and the invention will be described hereinafter in terms of the use of a scalpel. Furthermore, the surgical procedure need not involve actual removal of the tumour or other target within the brain, but may be purely exploratory. The term surgical procedure is thus used herein to denote any procedure in which a surgical instrument is caused to travel within the body of a mammal. Furthermore, a surgeon, but could be carried out by a skilled technician need not perform the procedure. The term surgeon is thus used herein to denote any person who operates the surgical instrument. In an extreme case it may be possible to program a computer to carry out the surgical procedure and such operation falls within the scope of the term surgeon and surgical procedure as used herein.

The vector information is provided by a visible image on the exposed surface of the brain. This image is formed by projecting one or more beams of light or other detectable radiation onto the exposed surface of the brain, preferably from sources of illumination which are laterally displaced from one another to provide separate images which converge or focus at the intermediate point. This image is preferably provided as a visible light image. However, other forms of detectable image may be used, for example fluorescent images or beams of other forms of radiation, eg gamma radiation, which can be detected by a suitable sensor. For convenience, the invention will be described hereinafter in terms of the use of visible light beams.

The image projected onto the exposed surface of the brain can take a wide range of forms. For example, the image can take the form of three or more beams of light directed to a focus at the intermediate point to which the tip of the surgical instrument is to be directed. Such beams may form a ring of individual spots of light upon the exposed surface of the brain until the intermediate point is exposed, at which point the beams of light are focussed and will form a single spot image. The extent of splay of the spots will provide the surgeon with a clear visual indication as to how far the focus point is below the surface of the brain. The extent to which the spots are offset from the tip of the surgical instrument will indicate the lateral direction in which the tip must be directed.

Other forms or combinations of images may be used if desired. For example, the image may be in the form of an holographic image of the relevant portion of the brain which displays blood vessels and other features to be avoided and the optimum path for the surgical instrument to follow. Alternatively, the images may be in the form of crosses which are superimposed upon one another at the focal point, arrows whose tips converge at the focal point or a combination of different forms of image which assist the surgeon in determining the direction and depth of the intermediate point below the exposed surface of the brain.

For convenience, the invention will be described hereinafter in terms of using a plurality of spot images which converge to form a single spot image at the intermediate point.

The spot images can be formed by any suitable form of illumination, for example a pea bulb, LED or a laser. The spot images may be formed by convergent beams which are themselves focussed at the intermediate point so that the diameter of the individual spot images also provides an indication of the depth of the intermediate point below the surface carrying the spot images. Alternatively, the beams of light may be collimated so that the spot images remain of substantially constant size and it is the diameter of the triangle or ring of spots on the exposed surface of the brain which indicates the depth of the intermediate point below the surface.

The sources of illumination forming the individual spots of the image may be laterally displaced from one another so that the individual spots do not overlap one another until the exposed surface onto which the image is projected is close to the intermediate point. However, it will usually be preferred that the sources of illumination are sufficiently close to one another so that they overlap to provide a highly illuminated portion which is centred upon the line of the path to the intermediate point. If desired, the individual spots may be of different colours so that the area of overlap produces an image having a different colour to provide enhanced contrast between this area and other areas of the illuminated image and the exposed surface of the brain.

For convenience, the invention will be described hereinafter in terms of laser beams of light which converge to a point at the focus of the beams and which overlap to provide an area of more intense illumination substantially centrally within the overall image. As indicated above, the invention may be applied to surgical procedures in which no microscope is used, for example in surgery on the spine. However, the invention is of especial application in cranial surgery where it is customary for the surgeon to observe what he is doing through a microscope and the invention will be described hereinafter in terms of the use of a microscope. If desired, the microscope may be provided with one or more rings or crosses engraved or otherwise formed in the sight path of the surgeon to assist in centring the field of view of the microscope upon the centre of the overlap in the illuminated image.

The lasers or other sources of illumination used to form the image on the exposed surface of the brain can be located at any suitable location from which they can project an image onto the brain which is not obscured or interrupted by other equipment. Thus, for example, the sources of illumination could be mounted on the ceiling of the operating theatre, for example as a ring of high intensity LEDS or lasers directed at the head of the patient. However, they may also be mounted upon a suitable free-standing column, on an arm carried by the operating table upon which the patient rests during the surgical procedure or on another support which can be stood on the floor of the theatre and positioned as required by the surgeon. Alternatively, the sources of illumination may be carried by the chassis of the microscope to be used by the surgeon, so that the sources of illumination follow the movement of the microscope during the surgical procedure. In a further alternative, the sources of illumination can be built into the headband often worn by surgeons when not operating using a microscope or into a chest plate worn by the surgeon.

For convenience, the invention will be described hereinafter in terms of a plurality of lasers carried upon the chassis of the microscope through which the surgeon observes the exposed brain and the tip of the scalpel. However, it will be appreciated that the sources of illumination could be located elsewhere and their illumination carried to the desired point of projection by glass or plastic optic fibres. Thus, a single high intensity laser mounted in a floor mounted console could direct its output beam at the exposed proximal end of a cable formed from individual optic fibres which lead to the chassis of the microscope. Individual beams from the distal ends of the individual fibres in the cable can then form the individual spot images on the exposed surface of the brain.

The lasers are to form an image upon the exposed surface of the brain which is focussed below the surface of the brain at the intermediate point which the surgical instrument tip is to reach on it path to the tumour. This will usually require that the position in space and the orientation of the individual lasers be established and related not only to the head of the patient but also to the computerised image of the optimum path to the tumour. Where the lasers are mounted upon the chassis of the microscope, the relative position of the lasers to one another and to reference points, for example LEDs, on the microscope chassis will be known. The position of the chassis relative to the fiducial markers carried by the patient's head, or by a reference arc clamping the patient's head in position, will be tracked by cameras or other fixed sensors in the operating theatre in the conventional manner and using conventional techniques for monitoring the movement of equipment in the theatre using a computer and relating that positional information to the computerised image of the brain of the patient. Other forms of sensing and computing the position of the microscope, and hence the sources of illumination, may also be used, for example inertial or laser ring gyroscopes. By these techniques, the computer will be permanently updated of the position of the lasers and their relative position to the patient's head.

Having determined the position of the lasers relative to the patient, it is then necessary to focus the light emitted from them at the desired intermediate point along the path to the tumour. The lasers can be mounted so that the direction of the beam of light they each emit can be directed as required, for example using screw threaded adjustment means. Where an optic fibre is used to convey light from a laser or other source of illumination, the distal end of the fibre can be flexed to achieve the desired direction of the light beam issuing from the end of the fibre. Alternatively, pressure applied to the side wall of the fibre may cause deflection of the light beam to a sufficient extent. In this way the beam of light from each laser carried by a static support can be focussed at a given intermediate point. Where a plurality of intermediate points are to be traversed during an operation, the static support can carry a turret which is rotated or otherwise moved to bring successive groups of lasers or optic fibres into operation to illuminate the exposed surface of the brain with different images according to the intermediate point to be next traversed.

Where the lasers are mounted upon the chassis of the microscope through which the surgeon is viewing the exposed surface of the brain and the tip of the surgical instrument, it will be necessary to adjust the focussing of the lasers to compensate for the movement of the microscope relative to the patient's skull. It is therefore preferred to mount the lasers upon mountings whose orientation can be continuously adjusted throughout the surgical procedure. For example, the lasers could be mounted upon a support plate whose orientation is adjusted by screw mechanisms operated by stepper motors or other means. A particularly suitable form of mounting is that in which pairs of threaded rods extend between threaded receptors carried at the apices of one support member to receptors carried at the apices of a second similarly configured support member. Each pair of rods from one receptor on one member extends to adjacent receptors on the other member. Upon rotation of the rods and/or receptors, the orientation and position of the members with respect to one another can be varied. Such a mechanism enables the individual lasers to be directed accurately under control of stepper motors.

In an alternative form of apparatus, a plurality of lasers or the free ends of optic fibres conveying light from a single laser, can be provided in a cluster mounted upon a base member, each laser or fibre being orientated at a different angle to the base member. Where two or more such clusters are mounted upon the chassis of the microscope, illumination of a laser or fibre in each cluster will combine to form an image which is focussed a given distance from the microscope. By selecting different lasers or fibres, images focussed at other points can be achieved. Such a structure can be designed so that individual lasers or fibres from each of the clusters focus at specified points and suitable switching of the lasers in the clusters, for example under computer controlled selection, can achieve the formation of a range of images to focus on selected intermediate points in a patient's skull.

In place of lasers where the direction of the light beam they emit is varied to focus upon different intermediate points, a focussing lens or a tilting mirror can be used to focus the light beam from a fixed focus laser. The movement of the lens or mirror to achieve the desired focal point for the light beams from the lasers can be achieved using any suitable mechanism, for example a stepper motor and screw threaded adjustments means. Alternatively, the beam from a laser can be deflected using an acoustic coupler and a suitable crystal upon which the acoustic coupler or transducer acts, for example a germanium crystal.

Such structures readily lend themselves to control by computers so that the light beams from the lasers can be accurately focussed upon the desired intermediate point below the exposed surface of the brain during movement of the microscope. Furthermore, such structures can be programmed so that the lasers can be re-focussed upon the next intermediate point to be traversed in following the path to the tumour. It is thus possible for the surgeon to identify desirable intermediate points in the path from the point of entry into the patient's skull to the tumour itself and to program those into the memory of a computer controlling the focussing of the light beams from the lasers. That computer can then interface with the computer, which need not be a different computer, relating the position of the microscope to the patient's skull and to the computerised images of the patient's brain during the surgical procedure so that the lasers are focussed upon the desired intermediate points throughout the procedure and compensate for relative movements between the microscope and the patient. Such programming and interfacing of the positional and vector information can be achieved using conventional computer and programming techniques.

The invention thus enables the surgeon to identify desirable intermediate points in the path from the start to the finish of a surgical procedure, to program those into the computer(s) so that during the procedure he is presented with a visible image on the exposed surface of the brain which guides the tip of the surgical instrument to the tumour. Thus, the method of the invention allows the surgeon to follow an image showing where to make the initial skin incision of the scalp of the patient, then the exact site and size for opening the bone of the skull, then to direct the surgical instrument to the tumour without the need to divert the surgeon's attention from the manipulation of the surgical instrument or to make complex topographical analyses of the information presented to him.

The invention can readily be applied to presently available computer-assisted surgical systems by providing one or more sources of illumination to generate a plurality of images on the exposed surface of the patient, a mechanism for focussing those images at a desired intermediate point in the path to the tumour or other location within the patient, which point lies below the exposed surface upon which the image is generated, and a mechanism for inter-relating the information generating and directing the images to the computerised image of the patient and the actual head or other structure of the patient upon which a surgical procedure is to be performed. The operation of the image generation and focussing and its inter-linking to the computerised image of the patient and the location of the patient can be achieved using conventional positional and vector analysis software and mechanisms as used in computer operated image gathering, processing and computer controlled surgery.

The invention has been described above in terms of relating the movement of the tip of the scalpel to an already established optimal path to the tumour in the brain. However, for other surgical procedures, for example the insertion of screws or other metal objects in orthopaedic surgery, it may be desirable to develop the optimal path as the procedure progresses. Thus, the optimal path can be determined by the surgeon by taking a number of images, for example X ray images using a C arm or ultrasonic images, to determine the position of the tip of the scalpel and the structure within the spine or other location at which the surgeon is operating. The surgeon can interrupt the surgical procedure to examine the direct or computerised images to assess the next stage of the procedure and determine the optimal path to be followed, for example to direct a drill forming a screw anchorage bore in a bone.

The invention has been described above in terms of an image, which is focussed on the desired intermediate point. However, it is within the scope of the present invention for the focus of the image to be moved between the tip of the surgical instrument and the intermediate point below the exposed surface of the brain. By presenting an image which moves between these two points, the surgeon is given a moving guide towards the desired intermediate point. Such a moving image can often give the surgeon an enhanced perception of the direction in which to move the tip of the scalpel. Such a moving image is also readily generated by computer control of the focussing of the sources of illumination using conventional programming techniques. If desired, the movement of the image between the exposed surface and the intermediate point can be carried out in a plurality of steps. For example, the image can be focused at 1 to 5 mm intervals along the desired path between the exposed surface and the intermediate point, so that the surgeon is effectively provided with a number of shorter paths to follow between the exposed surface and the intermediate point. Such sub-division of the path assists the surgeon to move the tip of the surgical instrument accurately along the desired path and also alerts the surgeon to any deviation from the desired path by directing the tip of the surgical instrument repeatedly to a point along the desired path. Furthermore, by sub-dividing the path to be followed into such small steps, the surgeon is present with a virtually continuous vector guide as to where next to direct the tip of the scalpel.

The invention has been described above in terms of the sources of illumination being mounted upon the chassis of a microscope. However, many surgical procedures are carried out without the use of a microscope. In such cases, the sources of illumination can be mounted on a fixed stand or other support. However, it is also within the scope of the present invention for the sources of illumination to be carried on a head band or chest plate worn by the surgeon. In such cases, the sources of illumination will move relative to the patient's head or body as the surgeon moves. It will then be necessary to provide means for tracking the movement of the surgeon's head or chest, for example using cameras or other sensors to monitor the spatial movement and position of LEDs carried by the head band or chest plate worn by the surgeon. Such detectors and their monitoring can use conventional equipment and techniques.

The invention has been described above in terms of providing the surgeon with guidance as to where next to direct the tip of the scalpel. However, as indicated above, means may also be provided for informing the surgeon as to where the tip of the scalpel is within the body of the patient. This can be achieved using any suitable technique, foe example by focusing the image from the sources of illumination at the exposed surface of the brain. This will require operation of the stepper motors or other drive means moving the lasers or other light sources and this movement can be detected by suitable means and used to provide a computer with information for assessing the location of the focal point of the microscope and relating that to the computerised image of the brain.

### DESCRIPTION OF THE DRAWINGS

The invention will now be described with respect to preferred embodiments thereof as shown in the accompanying drawings in which

FIG. 1 is a block diagram of a patient, the laser arrays, the microscope and the computer interfaces for controlling the focussing of the laser light beams;

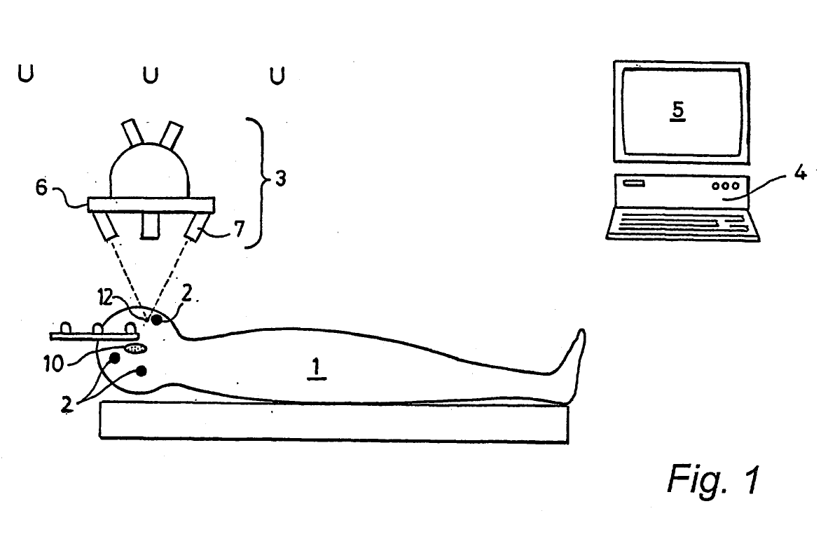

FIG. 2 shows in diagrammatic form the computerised image of a portion of the patient's skull showing a tumour and the optimal path from the point of entry into the skull to the tumour with a number of intermediate points that must be traversed along that path;

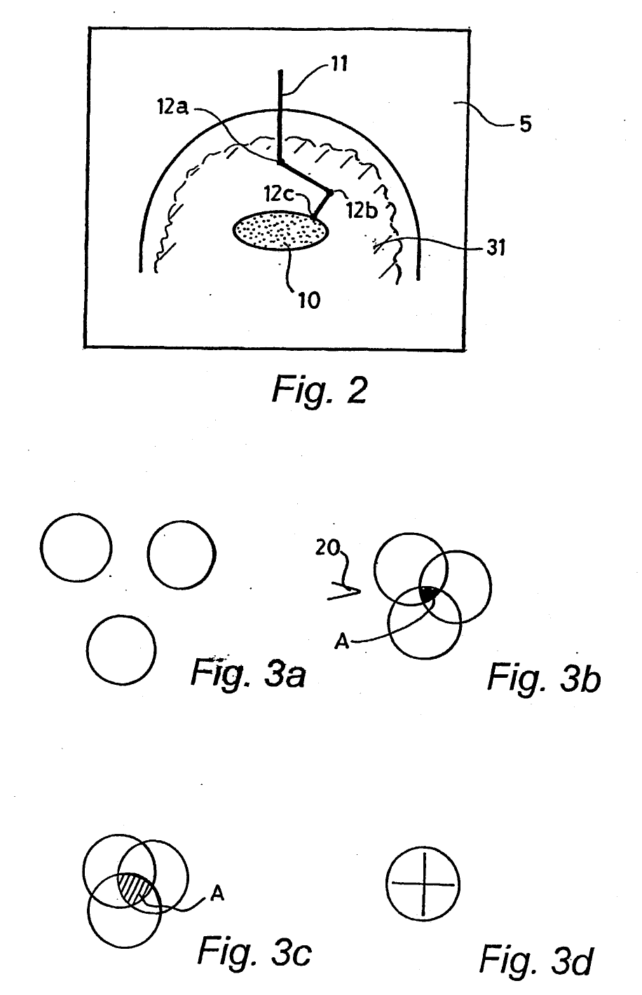

FIG. 3 is a series of diagrammatic views of the images projected onto the exposed surface of the brain of the patient as the scalpel tip approaches an intermediate point on the path to a tumour in the brain;

FIGS. 4 to 7 illustrate diagrammatically alternative forms for the mounting and focussing of the light beams onto the exposed surface of the brain of the patient.

### DESCRIPTION OF THE PREFERRED EMBODIMENTS

A patient 1 is placed upon an operating table with his head clamped securely so that it is retained in a fixed position for the surgical procedure. A series of metal studs 2 have already been secured to the patient's skull to enable the position and orientation of the skull to be detected by the surgeon touching the studs 2 with the tip of a marker wand and detecting the tip of the wand using conventional techniques. An arc carrying LEDs or other emitters is clamped in fixed orientation to the patient's head and the position of the arc detected and related to the position of the studs 2 using conventional techniques. This also enables a computerised image of the patient's head to be related to the physical position of the patient and to the position and orientation of the surgeon's microscope 3 in the conventional manner using the computer 4. The computerised image of the patient's head and of the desired path to the tumour is displayed on a VDU 5.

Initially the patient's head has been scanned to produce a plurality of X-ray or other images to detect and locate a tumour within the patient's brain. These images have been scanned into a suitable computer to generate a computerised image of the patient's brain and the tumour therein.

The operating table, microscope, studs, sensors and the image generation and detection software and computers controlling and generating them are of conventional structure and operation.

From the computerised images, the surgeon identifies the size and location of the tumour 10 and the optimum path 11 through the skull and to the tumour, minimising potential damage to other parts of the brain. This optimum path 11 identifies a number of intermediate points 12 at which the path needs to change direction. The surgeon identifies these and their position relative to the fixed datum points of the studs 2 in the patient's skull.

The chassis 6 of the microscope carries a plurality of lasers 7 which emit convergent beams of light which are directed at the exposed surface of the patient's skull or brain during the surgical procedure. The beams are focussed on the first intermediate point 12a along the path to the tumour. This focussing is achieved from a knowledge of the position of the microscope 3 relative to the fixed arc and the patient's skull and hence to the internal features of the patient's brain.

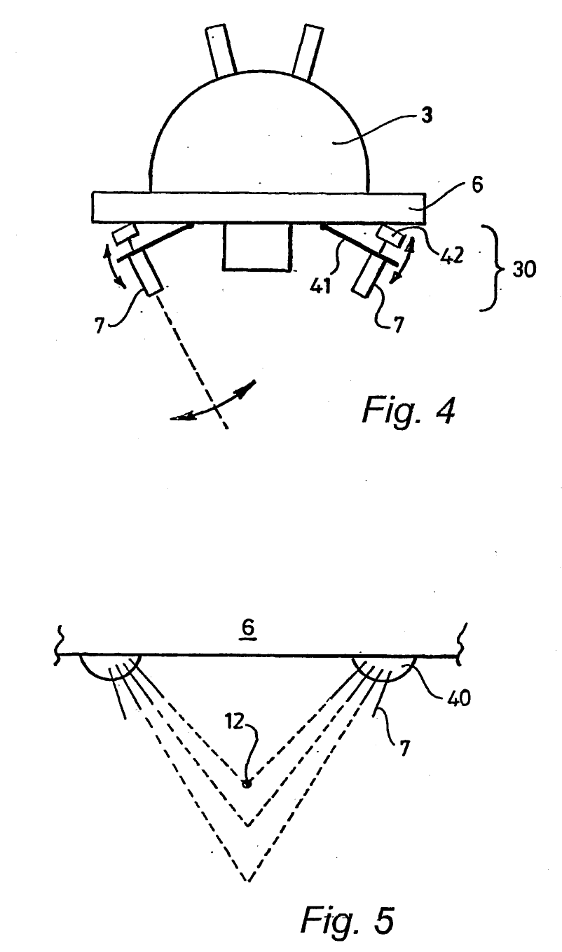

Initially, the beams of light will be focussed at a point below the exposed surface of the brain and will give rings of spots as shown in FIG. 3a. In this embodiment, the spots overlap one another to give an area of more intense illumination A. Where the lasers emit different coloured light beams, area A will be the sum of all those colours and may thus be of a contrasting colour. As the tip of the scalpel 20 is depressed into the tissue of the brain, or lobes of the brain are separated by paddles or retractors, the light beams fall upon a surface of the brain which is further along the path towards the tumour and hence closer to the intermediate point 12a. As a result the image changes and the spots overlap one another to a greater extent as shown in FIG. 3b. If the surgeon has deviated from the desired path to the tumour, the incision he has formed will no longer lie centrally within the light image, but will be off set as shown in FIG. 3c. The surgeon can readily detect this and make a suitable correction. However, since the light image is visible at all times upon the exposed surface of the brain, the surgeon is always guided along the correct path and the risk of deviation from the correct path is minimised. Once the surgeon has reached intermediate point 12a the light image will be a single dot, as shown in FIG. 3d. If desired, one of the lasers may emit a cruciform image which centres within one or more of the spot images from the other lasers. This aids detection of the intermediate point when the cross image fits within the circumference of the single dot image, as shown in FIG. 3d.

Point 12a may be upon the surface of the tumour and other intermediate points then define the extent of the tumour or the area to be excised by the surgeon. However, point 12a may be a point at which the optimum path to the tumour changes direction so as to avoid some structure within the patient's head. The surgeon will need to reset the lasers to focus upon the next intermediate point 12b. This can be done manually, for example by rotating a turret carrying a new set of lasers aligned at different angles to one another into position on the chassis of the microscope. However, it is preferred that the new point 12b be programmed into the mechanism controlling the focussing of the lasers so that the surgeon selects the next focus point by a suitable switch or keyboard input device. Alternatively, since the position of the microscope is being monitored by the sensors in the operating theatre, such a switch to the new focus point can occur automatically once the focal point of the microscope coincides with the focal point of the light beams at intermediate point 12a.

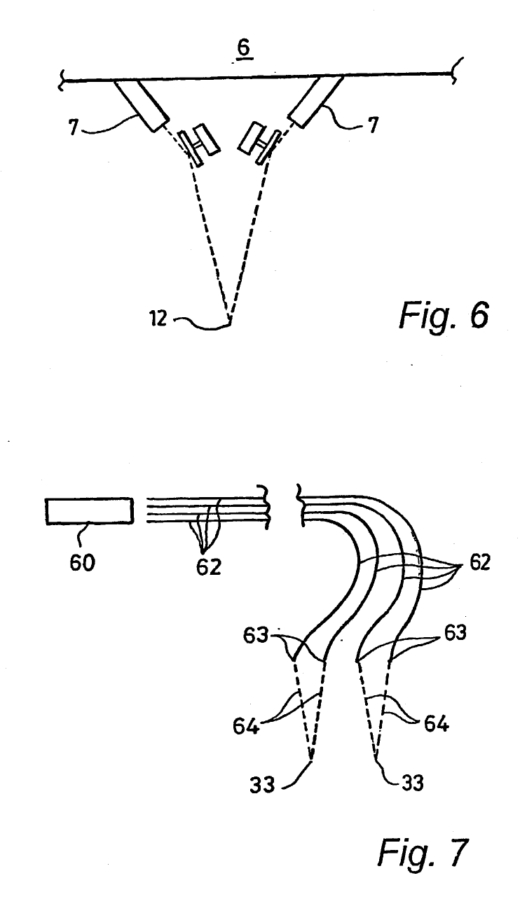

The lasers can be mounted individually upon a suitable directional mechanism 30 on the chassis 6 of the microscope 3. The mechanism 30 is operated under a suitable computer control as the surgeon moves the tip of the scalpel closer to the intermediate point and follows this movement with movement of the microscope. Alternatively, the lasers can be mounted in clusters 40, with the individual lasers directed at different angles as shown in FIG. 4. Suitable switching selects which lasers are actuated so as to direct the beams of light at intermediate point 12. Such a system will give stepwise changes in the direction of the light beams and hence minor variations in the focussing of the light beams about the position of point 12. In order to enhance the accuracy of the focussing of the light beams, as shown in FIG. 5, individual lasers 40 can be mounted on support plates 41 which are tilted about the axis of the light beam by stepper motors or-screw mechanisms 42 so that the beam of light from that LED is always directed at point 12. Alternatively, as shown in FIG. 6, the beam from a fixed laser can be directed by means of a tilting mirror. In the alternative form of device shown in FIG. 7, a single laser 60 illuminates the proximal ends of individual fibres 62 in an optic fibre cable. The illumination is carried along each fibre 62 to the distal ends 63 of the fibres 62 to provide separate beams of light 64 from each fibre. These can be focused, for example by flexing the distal end portions of the fibres using any suitable mechanism or by applying pressure to the side wall of the fibre, to focus the light beams at the intermediate point 33. If desired, one pair of fibres can be focused on a first intermediate point 33 and other pairs of fibres focused on a second or subsequent intermediate point 33 as shown in FIG. 7.

If desired, such mechanisms can be operated under the control of a computer so that the light beams are focussed on a point just below the exposed surface of the brain, and intermediate the exposed surface and the intermediate point on the optimum path to the tumour or other target. Typically, this will be just beyond, say 1 to 10 mms beyond, the focal point of the microscope so that the path to the tumour is formed from a series of small portions, say only 1 to 5 mms long. In this way the surgeon is presented with a series of closely spaced intermediate points and the risk of deviating from the desired path to the tumour is further reduced.

1. A method for proactively directing the movement of the operative tip of a surgical instrument during a surgical procedure being carried out on a mammal along a predetermined path within the body of the mammal from an exposed surface of the body of the mammal via at least one intermediate point along that path to a desired target within the body of the mammal, characterised in that the method comprises:
a. directing a detectable image onto the exposed surface of that portion of the mammal upon which the surgical procedure is being carried out, which image is focused at a point below the exposed surface, which point is one of the intermediate points to which the operative tip of the surgical instrument is to be transported along the path to the target during the procedure; and
b. causing the tip of the surgical instrument to follow the image to its focus and thus to that intermediate point along the path to the target, which creates a second exposed surface at that intermediate point.

2. A method as claimed in claim 1, characterised in that the path to the target incorporates a plurality of intermediate points and the method comprises the further steps of:
c. re-focusing the image below that second exposed surface at the next intermediate point in the path to the target; and
d. causing the tip of the surgical instrument to follow the image from the second exposed surface to the next intermediate point; and
e. repeating, if necessary, steps c and d until the target is reached.

3. A method as claimed in either of claims 1 or 2, characterised in that the image is a visible light image.

4. A method as claimed in any one of the preceding claims, characterised in that the image comprises a plurality of separate images which are directed to converge at the location of the intermediate point along the path to the target.

5. A method as claimed in any one of the preceding claims, characterised in that the image is formed by directing laser generated light beams at the exposed surface of the body.

6. A method as claimed in claim 4, characterised in that the images are substantially circular images on the exposed surface of the body which overlap to provide an area of increased intensity and/or colour along the path to the intermediate point to which the tip of the surgical instrument is to be transported.

7. A method as claimed in any one of the preceding claims, characterised in that the image is caused to translate between the exposed surface of the body and the intermediate point within the body to which it desired to transport the tip of the surgical instrument, whereby a moving image is provided which translates between the exposed surface and the intermediate point on the path to the target.

8. A method as claimed in any one of the preceding claims, characterised in that the desired path to the target has been determined by prior inspection of the mammal by x ray, magnetic resonance, computer aided tomography and/or infra sound techniques to provide an image of the relevant portion of the body of the mammal and this image and the co-ordinates of the intermediate points along the path are stored in a computer memory, whereby the image on the exposed surface of the body for the next intermediate point along the path to the target can be generated and displayed upon the body of the mammal as each intermediate point along the path is attained by the tip of the surgical instrument.

9. A method as claimed in any of the preceding claims, characterised in that the surgeon observes and directs the movement of the surgical instrument using a microscope whose movement in space is detected and monitored, whereby the position of the tip of the surgical instrument can be related to the desired path to the target.

10. A method as claimed in any one of the preceding claims, characterised in that the mammal is a human being.

11. A method as claimed in claim 11, characterised in that the surgical procedure is the treatment or removal of a tumour or other feature in the brain.

12. A method as claimed in claim 1, characterised in that the surgical procedure involves the affixing of an implant within the body of the mammal and the images on the exposed surface are used to guide a cutting or drilling device.

13. A method as claimed in claim 12, characterised in that the implant is to be secured to a bone or the skull of the mammal.

14. A method for proactively directing the movement of the operative tip of a surgical instrument within the body of a mammal as claimed in claim 1 substantially as hereinbefore described.

15. A method for proactively directing the movement of the operative tip of a surgical instrument within the body of a mammal substantially as hereinbefore described with respect to any one of the accompanying drawings.

16. Apparatus for use during a surgical procedure to be carried out on a mammal and for proactively guiding the tip of a surgical instrument to be used in that procedure along a predetermined path within the body of the mammal from an exposed surface on the body to a target within the body, via at least one intermediate point along that path, which apparatus is characterised in that it comprises:
a. a support member carrying a plurality of sources of illumination laterally displaced from one another whereby a detectable image can be formed upon that exposed surface of the mammal; and
b. a mechanism whereby those sources of illumination can be caused to focus at a series of intermediate points along the path which the tip of the surgical instrument is to follow between the exposed surface and the target; and
c. a computer programmed to relate the location in three dimensions of the body upon which the procedure is to be carried out to the locations of the intermediate points along the path to the target and to focus the sources of illumination at those intermediate points in succession along the path whereby a series of detectable images can be formed upon the exposed surface of the body to provide a sequence of guidance directions to successive intermediate points to be attained along the path between the exposed surface and the target, which images are focused below the said exposed surface.

17. Apparatus as claimed in claim 16, characterised in that the sources of illumination are sources of visible light beams.

18. Apparatus as claimed in either of claims 16 or 17, characterised in that the support for the sources of illumination is provided by the chassis of a microscope to be used in observing the exposed surface of the body of the mammal.

19. Apparatus as claimed in either of claims 16 or 17, characterised in that the support member is a static member, and the computer is programmed to relate the location in three dimensions of the support member to the location of the intermediate points.

20. Apparatus as claimed in either of claims 16 or 17, characterised in that the sources of illumination are carried by a mobile support member and the computer is programmed to relate the location in three dimensions of the support member to the location intermediate points.

21. Apparatus as claimed in any one of claims 16 to 20, characterised in that the computer is programmed to translate the images formed on the exposed surface of the body between the surface and the intermediate point next to be attained along the path to the target whereby a moving image is formed on the exposed surface of the body to provide a dynamic guidance image of the path between the exposed surface and that intermediate point.

22. Apparatus as claimed in any one of claims 16 to 21, characterised in that at least three images are to be formed on the exposed surface and to overlap one another whereby the path to the intermediate point is indicated by an area of increased intensity of illumination and/or of different colour.

23. Apparatus as claimed in any one of the preceding claims, characterised in that the desired path to the target has been determined by prior inspection of the mammal by x ray, magnetic resonance, computer aided tomography and/or infra sound techniques to provide an image of the relevant portion of the body of the mammal and this image and the co-ordinates of the intermediate points along the path are stored in the memory of the computer, whereby the image on the exposed surface of the body for the next intermediate point along the path to the target can be generated and displayed upon the body of the mammal as each intermediate point along the path is attained by the tip of the surgical instrument.

24. Apparatus as claimed in claim 16, substantially as hereinbefore described.

25. Apparatus according to claim 16 substantially as shown in and described with respect to any one of the accompanying drawings.
